(19) United States
(12) Patent Application Publication
(10) Pub. No.: US 2026/0191582 A1
(43) Pub. Date: Jul. 9, 2026

(54) MULTIFUNCTIONAL CATHETER DEVICES AND METHODS FOR DIAGNOSING AND TREATING HEART CONDITIONS

(71) Applicant: Mayo Foundation for Medical Education and Research, Rochester, MN (US)

(72) Inventors: Christopher V. DeSimone, Rochester, MN (US); Samuel J. Asirvatham, Rochester, MN (US); Christopher J. McLeod, Ponte Vedra Beach, FL (US); Jason A. Tri, Rochester, MN (US)

(21) Appl. No.: 19/129,961

(22) PCT Filed: Nov. 29, 2023
(86) PCT No.: PCT/US2023/081561

§ 371 Date: May 14, 2025

Related U.S. Application Data
(60) Provisional application No. 63/428,642, filed on Nov. 29, 2022.

(51) Int. Cl. A61B 18/14; A61B 18/00

(52) U.S. Cl. CPC A61B 18/1492; A61B 2018/00357; A61B 2018/00577; A61B 2018/00875; A61B 2018/1407; A61B 2018/1475

(57) ABSTRACT

Devices and methods can be used for diagnosing and treating medical disorders including heart conditions. For example, methods for cardiac mapping and pacing, as well as for treating ventricular fibrillation by delivering ablation and/or electroporation with high tissue specificity for destruction while minimizing collateral damage to critical structures of the heart and extracardiac structures. In some implementations, irrigation or a pharmacological agent is delivered simultaneously, before, and/or after with the application of the energy. In some embodiments, the devices described herein are capable of performing multiple functions from a single device. For example, in some embodiments the devices described herein can be used for two of more of at least the following modalities: cardiac mapping, cardiac pacing, tissue ablation, tissue electroporation, pharmacological agent delivery, irrigation, tissue stretching, force measurement, and temperature monitoring.

### CROSS-REFERENCE TO RELATED APPLICATIONS

This application claims the benefit of U.S. Provisional Application Ser. No. 63/428,642, filed Nov. 29, 2023. The disclosure of the prior application is considered part of the disclosure of this application, and is incorporated in its entirety into this application.

### BACKGROUND

### 1. Technical Field

This document relates to devices and methods for diagnosing and treating medical disorders including heart conditions. For example, among other things this document relates to devices and methods for cardiac mapping and pacing, as well as for treating ventricular arrhythmias such as ventricular fibrillation by delivering catheter-based ablation with radiofrequency energy and/or DC electroporation.

### 2. Background Information

Ventricular fibrillation (also referred to herein as “VF”) is a lethal rhythm that can result in sudden cardiac death (SCD). This is the number one cause of death-greater than all deaths from cancer in the United States combined. There is no cure for ventricular fibrillation that can lead to SCD-only treatments which are aimed at prevention of SCD such as drug therapy (which may be ineffective and fraught with side effects). ICD (“implantable cardiac defibrillator”) therapy is protective and could shock the patient back into normal rhythm, but also portends patients to ineffective shocks, inappropriate shocks, as well as post-traumatic stress disorder from receiving shock therapy. Radiofrequency (RF) ablation is limited in efficacy and issues with thermal ablation could lead to complications and unwanted tissue destruction. Although defibrillators, anti-arrhythmics, and other therapies provide an element of protection in select cases, sudden cardiac death remains a major worldwide health problem.

Electroporation is a technique that uses very brief pulses of high voltage to introduce multiple nanopores within the cells' wall in a non-thermal manner (unlike RF), specifically within the lipid bilayer of the cell membranes as a result of the change in electrical field. Depending on the voltage and frequency of pulsations used, these pores can be reversible (i.e., increase the permeability of these cell to chemotherapeutic agents and cellular “stunning”) and or irreversible (i.e., trigger cell death by the process of apoptosis or necrosis). Given the different composition of each cell-type membrane, electroporation can allow for a differential effect on different tissues.

The recording and analysis of intracardiac electrograms forms the basis for cardiac mapping. In some cases, cardiac mapping is performed with catheters that are introduced percutaneously into the heart chambers. The catheters can include electrodes that are used to record the endocardial electrograms. Recorded data of the catheter location and intracardiac electrogram at that location can be used to reconstruct in real-time a representation of the three-dimensional geometry of a portion of the heart.

Cardiac pacing involves electrical cardiac stimulation via one or more electrodes of a device to treat a bradyarrhythmia or tachyarrhythmia until it resolves, or until long-term therapy can be initiated. In some cases, the purpose of temporary pacing is to reestablish normal hemodynamics that are acutely compromised by a slow or fast heart rate. Finally, pacing maneuvers can be used to evaluate efficacy or completeness of ablation (threshold pacing), and varying electrical wavefronts to uncover still viable tissue.

### SUMMARY

This document describes devices and methods for diagnosing and treating medical disorders including heart conditions. For example, among other things this document describes devices and methods for cardiac mapping and pacing, as well as for treating ventricular fibrillation by delivering radiofrequency ablation and/or DC electroporation with high tissue specificity for destruction while minimizing collateral damage to critical structures of the heart and extracardiac structures.

In one aspect, this document describes a multi-functional catheter system that includes a delivery sheath defining a first lumen and a longitudinal axis; a catheter shaft; a plurality of electrode-carrying elements attached to and distally extending from a distal end portion of the catheter shaft; and a plurality of electrodes disposed on the plurality of electrode-carrying elements. The catheter shaft and the plurality of electrode-carrying elements are slidably disposable within the first lumen of the delivery sheath and thereby reconfigurable between: (i) a low-profile delivery configuration when the catheter shaft and the plurality of electrode-carrying elements are fully within the first lumen and (ii) a deployed configuration when the plurality of electrode-carrying elements distally extend from a distal end of the first lumen.

Such a multi-functional catheter system may optionally include one or more of the following features. The plurality of electrode-carrying elements may be configured to self-expand to define a conical shape when in the deployed configuration. The catheter shaft may define a second lumen. The multi-functional catheter system may also include a guidewire slidably disposable in the second lumen and distally extendable through and beyond the plurality of electrode-carrying elements. The multi-functional catheter system may also include at least one electrode attached to the delivery sheath. The multi-functional catheter system may also include at least one electrode attached to the catheter shaft. The multi-functional catheter system may also include a plurality of conjoining elements that each extend between distal tips of two electrode-carrying elements of the plurality of electrode-carrying elements to form an electrode loop. The plurality of conjoining elements may be slidably disposed within lumens of the plurality of electrode-carrying elements such that the width or area of the electrode loops are adjustable by tensioning or relaxing the plurality of conjoining elements.

In another aspect, this disclosure is directed to a method for treating a patient. The method includes advancing any embodiment of the multi-functional catheter described herein into the patient to position the plurality of electrode-carrying elements in a target region; and energizing at least some of the plurality of electrodes. The energizing provides an energy sufficient for ablation or electroporation of at least some tissue of the target region (e.g., reversible and/or irreversible electroporation).

Such a method for treating a patient using the multi-functional catheters described herein may optionally include one or more of the following features. The method may also include stretching, by the plurality of electrode-carrying elements, the at least some tissue of the target region. The stretching may occur simultaneously with the energizing. The energizing may include using at least one electrode attached to a single electrode-carrying element of the plurality of electrode-carrying elements as a cathode; and using at least one other electrode attached to the single electrode-carrying element of the plurality of electrode-carrying elements as an anode. The energizing may include using at least one electrode attached to a first electrode-carrying element of the plurality of electrode-carrying elements as a cathode; and using at least one electrode attached to a second electrode-carrying element of the plurality of electrode-carrying elements as an anode. The energizing may include delivering RF energy, ultrasound energy, light energy, and/or laser energy; and delivering pulsed DC energy. The method may also include delivering, via one or more of the plurality of electrode-carrying elements suction, irrigation, or a pharmacological agent to the target region. In some embodiments, the target region is a left ventricle, right ventricle, atria, and/or epicardium of the patient.

Particular embodiments of the subject matter described in this document can be implemented to realize one or more of the following advantages. Medical conditions such as ventricular fibrillation and others can be effectively treated using the devices and methods described herein. In some embodiments, the devices described herein are advantageously capable of performing multiple functions from a single device. For example, in some embodiments the devices described herein can be used to perform two of more of at least the following modalities: cardiac mapping, cardiac pacing, tissue ablation, tissue electroporation, pharmacological agent delivery, irrigation, tissue stretching, force measurement, defibrillation, changes in tissue impedance, and temperature monitoring. In some embodiments, ventricular fibrillation can be treated by ablation while preventing or reducing collateral damage to critical structures of the heart during the ablation procedure using the devices and methods described herein.

In some embodiments, the devices described herein include multiple slender, flexible electrode-carrying elements that can advantageously access otherwise hard to reach crevices and other anatomical areas anywhere within a heart chamber such as, but not limited to, the right and left ventricles.

In some embodiments, the uptake of a pharmacological agent to the tissue receiving the ablation treatment can be promoted using the methods and devices provided herein.

In some embodiments, a combination of two or more different types of radiofrequency ablation, ultrasound energy, laser energy, photo biomodulation, and/or DC pulsed-field electroporation energy can be strategically delivered using the devices and methods described herein. For example, in some embodiments radiofrequency (RF) energy can be delivered concurrently or sequentially with pulses of direct current (DC) energy. Such delivery of multiple energy types can be leveraged, as described further below, to enhance the overall effects provided by the devices and methods described herein.

In some embodiments, various medical conditions can be treated in a minimally invasive fashion using the devices and methods provided herein. Such minimally invasive techniques can reduce recovery times, patient discomfort, and treatment costs.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### BRIEF DESCRIPTION OF THE DRAWINGS

FIG. 1 is a schematic diagram of a heart that can undergo a mapping, pacing, thermal-based ablation such as radiofrequency and/or DC pulsed-electric field electroporation procedure using a catheter-based device in accordance with some embodiments provided herein.

FIG. 2 is a perspective view of a distal end portion of an example catheter-based mapping, pacing, ablation and/or electroporation system in accordance with some embodiments.

FIG. 3 is an end view (looking proximally) of the distal end portion of the catheter-based mapping, pacing, ablation and/or electroporation system of FIG. 2.

FIG. 4 is a perspective view of a distal end portion of another example catheter-based mapping, pacing, ablation and/or electroporation system in accordance with some embodiments.

FIG. 5 shows the distal end portion of the catheter-based mapping, pacing, ablation and/or electroporation system of FIG. 2 with the multi-electrode catheter contained within the delivery sheath and with a guidewire distally extending from the system.

FIG. 6 shows the arrangement of FIG. 5 with the distal end portion of the multi-electrode catheter extended distally out of the confines of the delivery sheath.

FIG. 7 is a perspective view of a distal end portion of another example catheter-based mapping, pacing, ablation and/or electroporation system in accordance with some embodiments.

FIG. 8 is another perspective view of the catheter-based mapping, pacing, ablation and/or electroporation system of FIG. 7.

FIG. 9 shows an entire example catheter-based mapping, pacing, ablation and/or electroporation system in accordance with some embodiments.

FIG. 10 shows an enlarged view of a handle portion of the system of FIG. 9.

Like reference numbers represent corresponding parts throughout.

### DETAILED DESCRIPTION

This document describes devices and methods for diagnosing and treating medical disorders including heart conditions. For example, among other things this document describes devices and methods for cardiac mapping and pacing, as well as for treating ventricular fibrillation by delivering ablation and/or electroporation with high tissue specificity for destruction while minimizing collateral damage to critical structures of the heart and extracardiac structures. In some implementations, irrigation or a pharmacological agent is delivered simultaneously, before, and/or after with the application of the energy. In some embodiments, the devices described herein are capable of performing multiple functions from a single device. For example, in some embodiments the devices described herein can be used for two of more of at least the following modalities: cardiac mapping, cardiac pacing, tissue ablation, tissue electroporation, pharmacological agent delivery, irrigation, defibrillation, tissue stretching, force measurement, impedance monitoring, and temperature monitoring.

While the devices and methods described herein are primarily described in the context of catheter ablation of the endocardium within a right and/or left ventricle to mitigate ventricular fibrillation, other bodily areas and medical conditions may be treated using the concepts provided. For example, the devices and methods described herein may be used to target the mid-myocardium and or epicardium. The devices can also be used in combination with ablation of cardiac and extra-cardiac ganglia within the body such as the stellate ganglia. Such bipolar ablation can be done from various vantage points including the aortic arch, trachea, neck, to target the cardiac ganglia, renal nerves, splanchnic nerves, stellate ganglion and chain, great vessel ganglia, vagus nerve, all in order to target ganglia in the mediastinum and abdominal cavities for maximum anti-fibrillation effect.

While the embodiments described herein may be described as providing specific types of ablation, it should be understood that a variety of ablation and/or electroporation techniques and energy sources are envisioned for use alone or in combination with any of the devices and methods described herein. For example, monopolar, bipolar, and/or biphasic ablation and/or electroporation techniques can be used. Ablation energy sources such as radiofrequency (RF), direct current (DC), alternating current (AC) in non-cardiac applications, cryogenics, hot solutions, and the like, and combinations thereof, can be used with the devices provided herein. In some embodiments, non-thermal pulsed field ablation/electroporation (“PFA”) is delivered using the devices and methods described herein. Such PFA can preferentially ablate certain targeted tissues with minimal effects on surrounding, non-targeted tissues. In some embodiments, both DC (e.g., for PFA) and RF electrodes can be advantageously used in combination on the devices provided herein. That is, RF electrodes may be included because they are well suited for delivering ablation energy, while DC electrodes may also be included because they are well suited for electroporation and/or as iontophoretic sources for driving pharmacological agents into tissue. The use of DC and RF electrodes in combination can thereby provide a device that provides the benefits of both types of electrodes.

In some embodiments, the electrodes for delivery of the ablation energy are located on the exterior surfaces of the ablation devices. In other embodiments, one or more central electrodes may be additionally located on a catheter or an inner central shaft of the device. In some embodiments, a combination of types of electrodes are included in a single device, as described further below.

Another embodiment can have spikes and/or spindles on the device that are arranged to wedge into the surrounding tissue such as myocardial tissue. In some embodiments, such spikes or spindles can be metallic and/or made of the same material as the electrodes and may function as electrodes. In some embodiments, one or more magnets may be embedded into the splines. Such one or more magnets may be configured to integrate to poles placed on the outside of the patient's chest (e.g., via a wearable vest). This would in turn allow the electrodes and splines to be in contact with the ventricle surface to helping to ensure contact between the splines and the patient's target tissue.

In some embodiments, the devices described herein can have electrodes for recording and/or for pacing, both proximally and distally, as well as along the catheter device's length and/or spline's length. This arrangement can advantageously enable the use of algorithms that employ impedance measurements and electrogram-derived signals to preferentially deliver dosages of the ablation and/or electroporation energy. In some embodiments, such an algorithm will include checking the impedance and electrograms with machine learning to determine when the Purkinje signal or targeted signal has been eliminated. This targeted signal may be set via the electroporation generator or electrogram recording system. This algorithm may also alter the electrode delivery sequence, amount of energy delivered, pulse duration, and/or the number of energy pulses delivered in real time.

In some embodiments, the devices described herein include a structure of multiple elongate elements that are attached to or disposed on a distal end portion of a catheter. The structure can be self-expandable (e.g., made from a super-elastic material such as, but not limited to, nitinol with shape memory) and can have one or more electrodes disposed on each of the elongate elements. In some embodiments, to make the framework, a hypo tube, which is initially tubular, is laser cut, expanded, and shape set into a desired configuration.

In some embodiments, provisions for the delivery of suction/aspiration and/or a liquid pharmacological agent for enhancement of the ablation/electroporation treatment and/or for the prevention or reduction of stenosis and neointimal hyperplasia are contemplated. For instance, the drug paclitaxel is an example of one type of an antimitotic pharmacological agent that can be delivered to the tissue undergoing ablation to prevent or reduce fibrosis and stenosis of the tissue. Paclitaxel can be used beneficially because of its rapid uptake and prolonged retention. In some implementations, paclitaxel can be delivered in 3% saline (or similar hypertonic solution) to enhance further its uptake and retention. While paclitaxel is provided as an example, other pharmacological agents can also be used. In other implementations, a high-energy DC shock (e.g., about 100V to 3,000V or about 2 to 250 Joules) can be applied to the tissue during and/or after exuding the agent to effectively push the agent into the tissue.

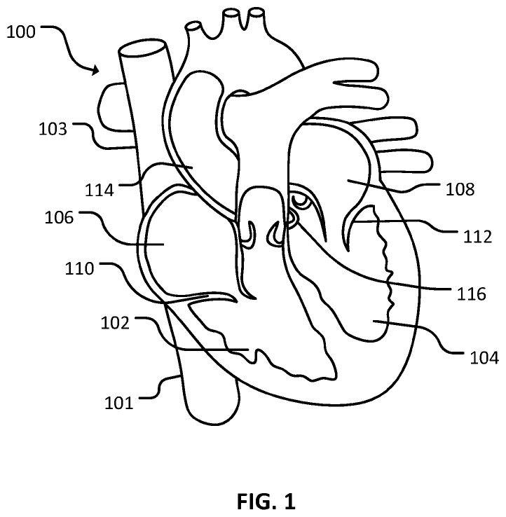

Referring to FIG. 1, a heart 100 includes a right ventricle 102, a left ventricle 104, a right atrium 106, and a left atrium 108. A tricuspid valve 110 is located between right atrium 108 and right ventricle 102. A mitral valve 112 is located between left atrium 108 and left ventricle 104. A semilunar or aortic valve 116 is located between left ventricle 104 and aorta 114. The aorta 114 conveys oxygen rich blood from the heart 100 to the body. An inferior vena cava 101 and superior vena cava 103 return oxygen depleted blood to the right ventricle 102.

Right ventricle 102 and/or left ventricle 104 can include Purkinje tissue. Purkinje fibers can be located on or in the ventricular walls of the heart and are specialized conducting fibers that allow the heart's conductive system to create synchronized contractions to maintain a consistent heart rhythm. Purkinje fibers can be superficial in right ventricle 102 and/or left ventricle 104. In some cases, there can be millions of Purkinje fibers. Purkinje fibers can also initiate tachyarrhythmias, such as those that cause ventricular fibrillation. These tissues may also be critical in maintenance of VF. Thus, modulation and/or ablation of critical segments of this tissue may render a person free of VF inducibility or result in an increase in a VF threshold for sustaining/maintaining this rhythm.

As described further below, devices and methods for mapping and/or administering pacing, ablation, and/or electroporation to locations of the heart 100 such as, but not limited to, the right ventricle 102 or the left ventricle 104 are provided herein. In some embodiments, bipolar ablation and/or electroporation can be delivered endocardially and/or epicardially. Moreover, using the provided devices and methods for administering electroporation, the Purkinje fibers can be targeted. Additionally, the ventricular myocardium can be targeted in the heart, such as the moderator band, right and left papillary muscles, the right and left septum of the ventricle, false tendons, etc. Furthermore, both Purkinje and ventricular tissue can both be targeted to have the desired effect of destroying tissue to eliminate VF and/or render a heart unable to go back into VF.

In some cases, using the devices and methods described herein, hemodynamic support can be optionally utilized during the procedure. For example, some of the methods described herein can optionally include the use of either a Left ventricular assist device (LVAD), Extracorporeal membrane oxygenator (ECMO), intraaortic balloon pump, or cardiopulmonary bypass (CPB) system to provide adequate hemodynamic support and oxygenation of the blood to permit safe and effective continuous mapping and ablation of VF for patient safety and feasibility. In some embodiments, the catheter(s) described herein may be integrated into the distal end of hemodynamical support devices to allow an integrated treatment and support system.

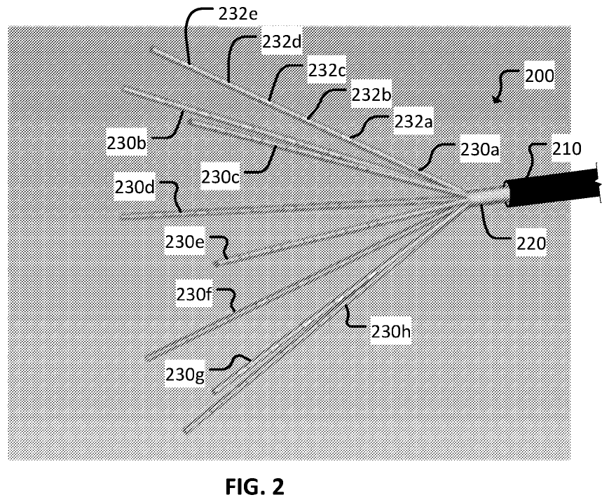

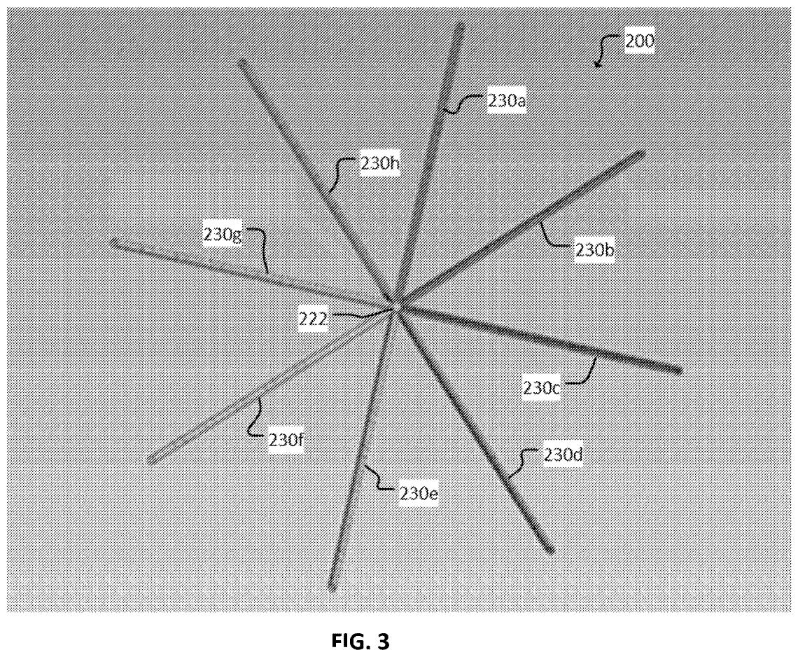

FIGS. 2 and 3 illustrate a first example multi-spline, multi-electrode, all-in-one mapping, pacing, ablating and/or electroporation catheter system 200 (hereinafter “catheter system 200”). The catheter system 200 includes a delivery sheath 210, a catheter shaft 220, and multiple slender electrode-carrying elements that distally extend from the distal end of the catheter shaft 220. In the depicted embodiment, the catheter system 200 includes eight of the electrode-carrying elements 230a, 230b, 230c, 230d, 230e, 230f, 230g, and 230h (collectively referred to hereinafter as “electrode-carrying elements 230a-h”). The electrode-carrying elements 230a-h distally terminate at free ends in the depicted embodiment. The lengths of the electrode-carrying elements 230a-h enable coverage of the heart ventricles from base to apex. This structure can also allow the distal end portions of the electrode-carrying elements 230a-h to advantageously access crevices within the chambers of the heart, such as in the ventricles.

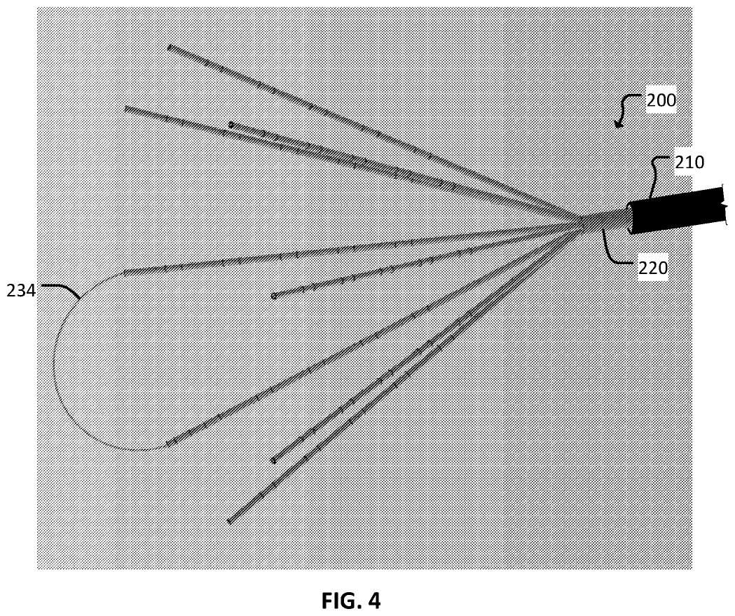

The catheter shaft 220, with its attached electrode-carrying elements 230a-h, are slidably disposed within a longitudinal lumen defined by the delivery sheath 210. Accordingly, a clinician who is operating the catheter system 200 to treat a patient can manipulate the catheter shaft 220 (outside of the patient), relative to the delivery sheath 210, to either distally extend (deploy) the catheter shaft 220 and electrode-carrying elements 230a-h from the distal tip of the delivery sheath 210 (as depicted in FIG. 2), or to proximally pull the catheter shaft 220 and electrode-carrying elements 230a-h into the low-profile confines of the delivery sheath 210 (as depicted in FIG. 4). In the depicted embodiment, when the electrode-carrying elements 230a-h transition from being contained within the low-profile confines of the delivery sheath 210 to being deployed, the electrode-carrying elements 230a-h elastically self-expand to the splayed configuration as depicted in FIGS. 2 and 3.

In some embodiments, a control system (e.g., a handle with various actuators) can be provided for the clinician to operate the catheter system 200 outside of the patient. In some such embodiments, an actuator can be included on the handle by which the clinician operator can move the catheter shaft 220 and electrode-carrying elements 230a-h proximally and distally relative to the delivery sheath 210. In some embodiments, the actuator can include markings and/or detents to identify particular relative orientations between the catheter shaft 220 and electrode-carrying elements 230a-h versus the delivery sheath 210. For example, such markings and/or detents can be included to indicate relative orientations such as, but not limited to, fully extended, fully retracted, partially extended, and the like.

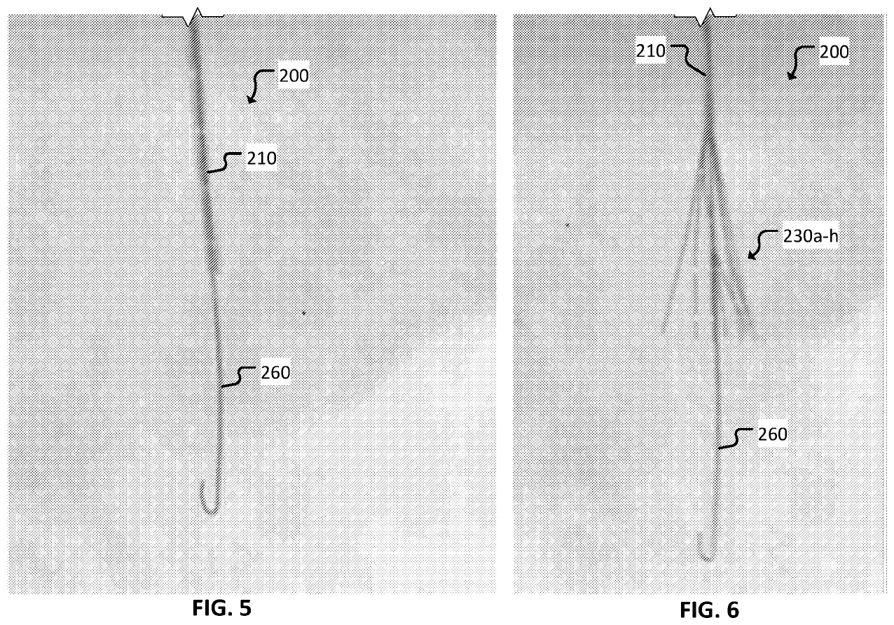

The delivery sheath 210 provides a low profile for trans-vascular delivery of the catheter system 200. In some embodiments, the delivery sheath 210 can include one or more pull wires by which a distal portion of the delivery sheath 210 is steerable, deflectable, or articulable by the clinician operator. In some embodiments, the delivery sheath 210 is steerable in a single plane. Alternatively, in some embodiments the delivery sheath 210 can be steerable in two different planes. In some embodiments, one or more radiopaque markers can be included on the delivery sheath 210 to enable the clinician operator to use fluoroscopic guidance while advancing the delivery sheath 210. In some embodiments, the delivery sheath 210 serves as a large return electrode (in conjunction with the electrode-carrying elements 230a-h). In some embodiments, a guidewire (e.g., refer to FIGS. 4 and 5) can be advanced to a target location and the catheter system 200 can be advanced over the guidewire.

Still referring to FIG. 1, the distal portion of the catheter system 200 can be navigated to the target locations within the heart 100 in various ways. For example, to access the left ventricle 104, in one example the catheter system 200 can be percutaneously inserted into a femoral vein of a patient and then navigated to the inferior vena cava 101. From the inferior vena cava 101, the catheter system 200 can be advanced into the right atrium 106. From the right atrium 106, the catheter system 200 can be advanced (via a puncture or opening in an atrial septum) into the left atrium 108. From the left atrium 108, the catheter system 200 can be advanced across the mitral valve 112 and into the left ventricle 104. With the distal end portion of the catheter system 200 within the left ventricle 104, the clinician operator can move the catheter shaft 220 and electrode-carrying elements 230a-h relative to the delivery sheath 210 to deploy the electrode-carrying elements 230a-h to the operative configuration shown in FIGS. 2 and 3.

In another example, the distal portion of the catheter system 200 can be navigated to the left ventricle 104 by percutaneous access to a femoral artery and using a retro-aortic approach into the left ventricle 104.

In one example for accessing the right ventricle 102, the catheter system 200 can be percutaneously inserted into a femoral vein of a patient and then navigated to the inferior vena cava 101. From the inferior vena cava 101, the catheter system 200 can be advanced into the right atrium 106. From the right atrium 106, the catheter system 200 can be advanced across the tricuspid valve 110 and into the right ventricle 102.

As briefly stated above, in some cases the clinician can choose to first install a guidewire prior to advancing the catheter system 200 within the vasculature and heart 100 of the patient. FIG. 3 shows a central guidewire lumen 222 that is longitudinally defined by the catheter shaft 220. The guidewire lumen 222 can slidably receive a guidewire. Accordingly, the catheter system 200 can be slidably advanced over such a guidewire. Once safely at the desired location, the guidewire can be left in place to add stability and maneuverability around the heart chamber. It can also be withdrawn inside the catheter shaft 220 to allow for full maneuverability of the electrode-carrying elements 230a-h.

Referring also to FIG. 4, in some embodiments the catheter system 200 can include one or more conjoining elements 234. Such a conjoining element 234 comprises a wire that slidably extends within two of the flexible electrode-carrying elements and between the distal tips of the flexible electrode-carrying elements. In the depicted embodiment, two of the flexible electrode-carrying elements are conjoined using a conjoining element 234, while the other flexible electrode-carrying elements extend singularly from the catheter shaft 220. However, that is not a requirement in all embodiments. That is, in some embodiments two of the conjoining elements 234 can be included as part of the catheter system 200. The two conjoining elements 234 can conjoin two pairs of the flexible electrode-carrying elements (i.e., a first conjoining element 234 that conjoins a first pair of the flexible electrode-carrying elements and a second conjoining element 234 that conjoins a second pair of the flexible electrode-carrying elements). The remaining flexible electrode-carrying elements extend singularly from the catheter shaft 220.

In some embodiments, three of the conjoining elements 234 can be included as part of the catheter system 200. The three conjoining elements 234 can conjoin three pairs of the flexible electrode-carrying elements (i.e., a first conjoining element 234 that conjoins a first pair of the flexible electrode-carrying elements, a second conjoining element 234 that conjoins a second pair of the flexible electrode-carrying elements, and a third conjoining element 234 that conjoins a third pair of the flexible electrode-carrying elements). The remaining flexible electrode-carrying elements extend singularly from the catheter shaft 220.

The conjoining elements 234 can be manually tensioned and relaxed by a clinician to control the size and shape of the loop defined by the conjoining element and its corresponding pair of flexible electrode-carrying elements.

Referring also to FIG. 5, here the catheter system 200, with the catheter shaft 220 and electrode-carrying elements 230a-h (not visible) contained within the delivery sheath 210 in the delivery configuration, is shown on a J-wire guidewire 260. Such a J-wire guidewire 260 can be used for safe introduction of the catheter system 200 across valves and other sensitive anatomical structures.

FIG. 6 shows the same arrangement as FIG. 5 but with the delivery sheath 210 pulled back so that the electrode-carrying elements 230a-h have self-expanded to their deployed configurations.

Still referring to FIGS. 2 and 3, in the depicted embodiment there are eight of the electrode-carrying elements 230a-h. However, in some embodiments two, three, four, five, six, seven, nine, ten, eleven, twelve, or more than twelve of the electrode-carrying elements can be included. The electrode-carrying elements 230a-h can be configured with electrodes in the same way as each other, or one or more of the electrode-carrying elements 230a-h can be configured with electrodes in a way that is different from the other electrode-carrying elements 230a-h.

In the depicted embodiment, each of the electrode-carrying elements 230a-h includes five electrodes that are spaced apart from each other along the length of the electrode-carrying elements 230a-h. For instance, the example electrode-carrying element 230a includes a first electrode 232a, a second electrode 232b, a third electrode 232c, a fourth electrode 232d, and a fifth electrode 232e (the electrodes of all of the electrode-carrying elements 230a-h are collectively referred to hereinafter as the “electrodes 232”). In some embodiments, one, two, three, four, six, seven, eight, nine, ten, or more than ten of the electrodes can be included on a single one of the electrode-carrying elements 230a-h.

The electrodes 232 of the catheter system 200 can be used in multiple modes. First, in some embodiments the electrodes 232 of the catheter system 200 can be used for mapping. Second, in some embodiments the electrodes 232 of the catheter system 200 can be used for pacing. Third, in some embodiments the electrodes 232 of the catheter system 200 can be used for delivery energy for ablation or electroporation (e.g., non-thermal reversible electroporation or irreversible electroporation). In some embodiments, the electrodes 232 of the catheter system 200 can be used for all such modes. In some embodiments, some of the electrodes 232 can be used for one mode and others of the electrodes 232 can be used for one or more other modes.

All of the electrodes 232 can be operated individually independent from each other in some embodiments. In other examples, two or more of the electrodes 232 can be configured to function together. For instance, using the example context of energy delivery, in some embodiments all the electrodes along an individual electrode-carrying element (e.g., the electrodes 232a-e on the electrode-carrying element 230a) can be operated in unity to all function in the same manner (e.g., all as anodes or all as cathodes). In such a case, in some embodiments all the electrodes of an adjacent individual electrode-carrying element (e.g., the electrode-carrying elements 230b or the electrode-carrying elements 230h; refer to FIG. 3) can be operated to function as cathodes (when the electrodes 232a-e are anodes) or as anodes (when the electrodes 232a-e are cathodes). It should be understood that this arrangement is just one example to illustrate that the electrodes 232 can be flexibly operated in many different manners and configurations as desired by the clinician operator of the catheter system 200.

In some embodiments, one or more of the electrode-carrying elements 230a-h can be configured with a single, continuous electrode extending along all or a majority of the longitudinal length of the particular one or more of the electrode-carrying elements 230a-h. Such an electrode-carrying element with the single long electrode can be operated as an anode or a cathode when the catheter device 200 is being operated in an energy delivery mode.

In some embodiments, one or more of the electrodes on an individual electrode-carrying element can be operated as an anode while one or more of the other electrodes on the same electrode-carrying element can be operated as a cathode. The operation of the individual anodes/cathodes can be timed such that a desired particular sequence or pattern of energy delivery can result.

The proximal end of the catheter shaft 220 (i.e., electrical wires proximally extending from the electrodes 232) can be connected to a controller and/or system of various types. For example, in the context of mapping, in some embodiments the proximal end of the catheter shaft 220 can be connected to a three-dimensional imaging system of a cardiac mapping system. In the context of energy delivery for pacing, in some embodiments the proximal end of the catheter shaft 220 can be connected to a cardiac pacing controller system. In the context of energy delivery for ablation and/or electroporation in some embodiments the proximal end of the catheter shaft 220 can be connected to an ablation energy source and controller (e.g., an RF, DC, ultrasound, laser, and/or AC generator/controller system not shown) which are located external to the patient. In such a case, the electrodes 232 can be energized with ablation and/or electroporation energy from the generator/controller system to initiate the modulation of target neural and/or muscle fibers/tissues in and/or around the target tissue.

In some examples, while the electric field for ablation or electroporation is being applied, a liquid pharmacological agent, irrigation, suction, or tissue stretching can be concurrently delivered to the tissue via the catheter system 200. Accordingly, in some embodiments one or more of the electrode-carrying elements 230a-h can include one or more ports through which a liquid pharmacological agent, irrigation, or suction can be applied. Such ports can be located between the electrodes 232, through the central lumen, or at the distal tip of the electrode-carrying elements 230a-h, for example.

The delivery of a liquid pharmacological agent can provide advantageous results in some cases. For example, delivering the agent prior to the ablative energy can provide iontophoresis-like action to drive the agent farther into the tissue. In another example, delivering the ablative energy prior to the pharmacological agent can provide some electroporative disruption of the endothelial cell-to-cell junction, thus promoting the agent delivery. In some implementations, a repetitious cyclic delivery of ablative energy and the pharmacological agent can thereby further enhance uptake of the agent. In some implementations, the pharmacological agent can have an ionic base so as to optimize the ablative energy's ability to get the agent beyond the endothelium of the tissue.

Paclitaxel is an example of one type of antimitotic pharmacological agent that is well suited for this application. This technique of coordinating the delivery of paclitaxel with the ablation process can prevent or reduce the occurrence of fibrosis, stenosis, and neointimal hyperplasia of the tissue undergoing ablation. Calcium and other types of biologic or non-biologic agents can also be delivered in some embodiments.

In some embodiments, one or more types of sensors can be located on one or more of the electrode-carrying elements 230a-h. For example, in some embodiments temperature can be measured using thermistors on the electrode-carrying elements 230a-h. In some embodiments, one or more sensors for pH measurements can be included on one or more of the electrode-carrying elements 230a-h. In another example, in some embodiments one or more sensors for force measurements can be included on one or more of the electrode-carrying elements 230a-h.

The catheter system 200 includes the one or more flexible electrode-carrying elements 230a-h that can “fan out” when deployed. The extent of the fanning out of the one or more flexible electrode-carrying elements 230a-h (e.g., the area defined by the tips of the electrode-carrying elements 230a-h) is controllable by the position of the catheter shaft 220 relative to the delivery sheath 210. That is, the one or more flexible electrode-carrying elements 230a-h can be allowed to fan out in a wider pattern by moving the catheter shaft 220 distally relative to the delivery sheath 210 and can be constrained to a smaller pattern by moving the catheter shaft 220 proximally relative to the delivery sheath 210.

In some embodiments, a return electrode for any of the electrodes of the electrode-carrying elements 230a-h can be placed in the epicardial space (to ablate across the LV wall) or in the RV (in order to ablate across the septum). The specificity of ventricular versus HPS ablation and vice versa can be modulated by varying the delivery of pulsed-electric fields from the catheter system 200 with a plurality of delivery protocols and parameters.

In some embodiments, portions of catheter system 200 can be enhanced to provide radiographic visualization of the position and orientation of the catheter system 200. For example, some embodiments include a loop of radiopaque material (e.g., titanium, tungsten, barium sulfate, zirconium oxide, and the like) around the distal tip of the delivery sheath 210 to allow for precise positioning and verification before proceeding with the procedure. Moreover, in some embodiments, one or more radiopaque markers can be positioned on one or more of the electrode-carrying elements 230a-h.

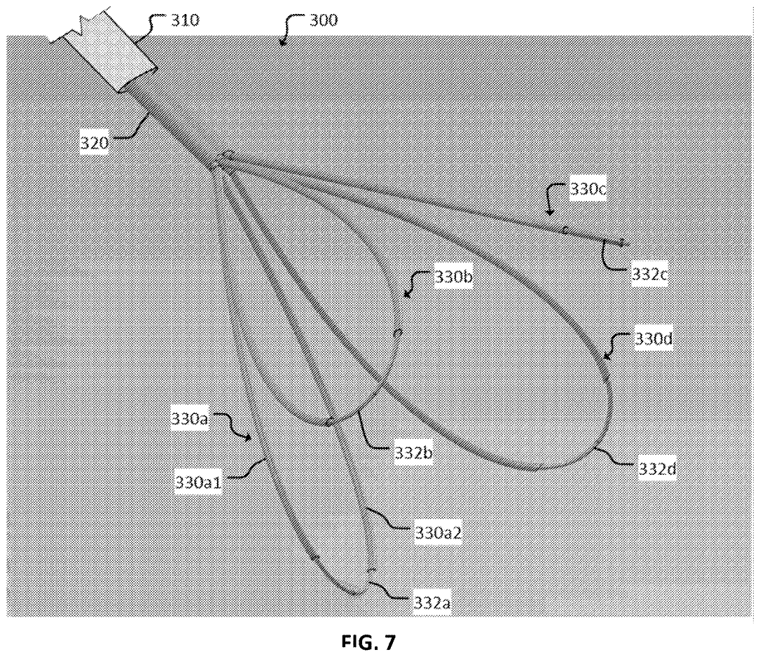

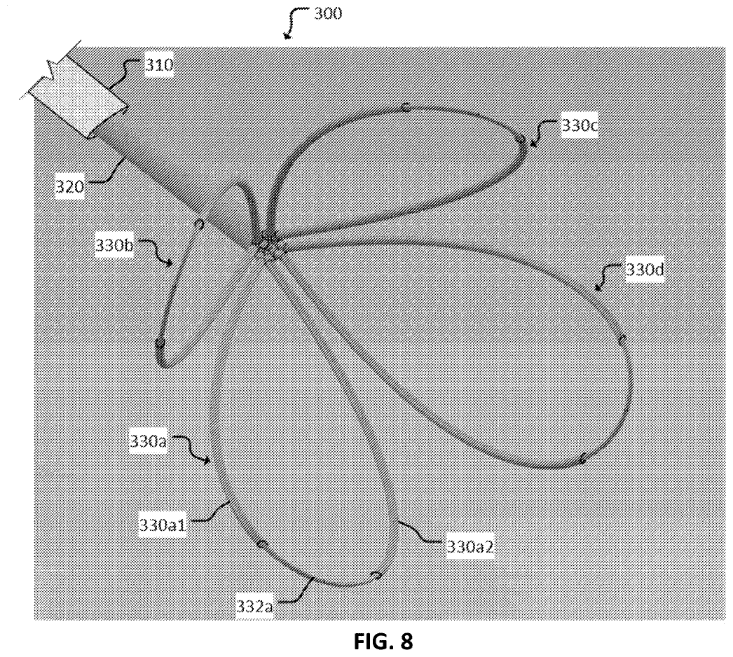

FIGS. 7 and 8 illustrate another example multi-spline, multi-electrode, all-in-one mapping, pacing, ablating and/or electroporation catheter system 300 (hereinafter “catheter system 300”). The catheter system 300 includes a delivery sheath 310, a catheter shaft 320, and, in this example, four electrode loops 330a, 330b, 330c, and 330d (collectively referred to hereinafter as “electrode loops 330a-d”). In a manner analogous to the catheter system 200 described above, the catheter shaft 320 and electrode loops 330a-d are slidably disposed in the longitudinal lumen defined by the delivery sheath 310. Accordingly, the electrode loops 330a-d are configurable in a low-profile arrangement within the delivery sheath 310 and are self-expandable to the fanned-out deployed arrangement as depicted in FIGS. 6 and 7.

The example electrode loop 330a includes a first flexible electrode-carrying element 330al, a second flexible electrode-carrying element 330a2, and a conjoining element 332a that slidably extends within the flexible electrode-carrying elements 330al and 330a2 and between the distal tips of the flexible electrode-carrying elements 330al and 330a2. In the depicted embodiment, the electrode loops 330b, 330c, and 330d are configured the same as the electrode loop 330a. However, that is not a requirement in all embodiments. That is, in some embodiments one or more singular flexible electrode-carrying elements (e.g., like the flexible electrode-carrying elements 230a-h as described above) can be included as part of the catheter system 300.

While not shown in FIGS. 7 and 8, it should be understood that the electrode loops 330a-d can include one or more electrodes along the longitudinal lengths of the flexible electrode-carrying elements (in a manner that is analogous to the flexible electrode-carrying elements 230a-h as described above). Such electrodes can be operated (individually and/or jointly) in any of the manners described above to provide mapping, pacing, ablation, and/or electroporation in any desired pattern, sequence, or configuration using the catheter system 300.

One difference between the catheter system 200 and the catheter system 300 is presence of the conjoining elements 332a-d that extend between two of the flexible electrode-carrying elements to form the electrode loops 330a-d. In some embodiments, the conjoining elements 332a-d are made of a super-elastic material such as, but not limited to, Nitinol.

The conjoining elements 332a-d are slidably disposed in one or more of the lumens of the two respective flexible electrode-carrying elements and proximally extend to the control handle that is manipulated by the clinician. Accordingly, by tensioning or relaxing the conjoining elements 332a-d the clinician operator can control the width or area of the individual electrode loops 330a-d.

In some embodiments, the conjoining elements 332a-d can function as an electrode. For example, in some embodiments the conjoining elements 332a-d can function as anode and/or cathode and can operate in conjunction with the electrodes on the electrode loops 330a-d.

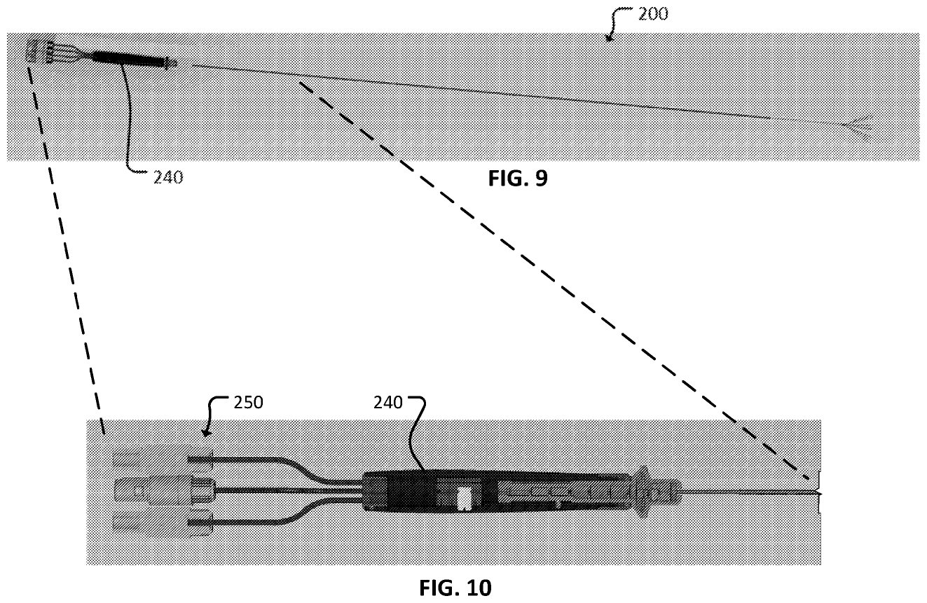

FIG. 9 shows an entirety of the example multi-spline, multi-electrode, all-in-one mapping, pacing, ablating and/or electroporation catheter system 200.

FIG. 10 shows an enlarged view of a control handle 240 of the catheter system 200. A clinician can manipulate and control the catheter system 200 (e.g., the positioning, configuration, mapping, impedance measuring, and energy delivering) using the control handle 240 outside of the patient's body. One or more connectors 250 (e.g., three connectors 250 in this example) extend from the control handle 240 for connection to other various devices such as, but not limited to, an ablation energy source, a mapping system, a control system, an impedance measurement system, and the like,

### Example Methods of Using the Multi-Functional Catheter Systems

The inventors envision many different methods for using the multi-functional mapping, pacing, ablating and/or electroporation catheter systems described herein. For example, in some cases the single catheter system can be used to deliver ablation, then pacing, and then mapping to determine whether the ablation was sufficient, or whether further ablation energy should be delivered.

In another example, the single catheter system can be used to deliver reversible electroporation to a target area to confirm that ablation delivered to that area will be effective for treating VF. If the test using reversible electroporation is effective, then ablation energy can be delivered to the same area to permanently treat the VF.

### ADDITIONAL OPTIONAL FEATURES, EMBODIMENTS, AND USES

The systems described herein can be used for minimal and maximal mapping of the ventricles, His-Purkinje system, and intracavitary dimensions that are complex within the ventricle, valve apparatus, false tendons, papillary muscles, moderator band, proximal and distal His-Purkinje fibers and branches.

The sizing of electrodes can all be the same (such as 1-5 mm each) or can vary along the catheter splines. The energy delivery can be selective based on changing the parameters of energy delivery for electroporation. The pairing of electrodes can be varied by electrical connection and disconnection in order to determine which pair of electrodes are active or turned off to allow for selective mapping, selective pacing, and selective ablation with electroporation and/or radiofrequency. Some of the splines can invade critical regions of the cavity that are otherwise essentially inaccessible, this is with the use of an extending finger/snake helical extension can also be used to wrap around the base of the papillary muscles, as well as provide a means to get to the septal and lateral aspects of the ventricle.

The proximal pairs of electrodes and distal pairs of electrodes may include protective shielding in order to prevent damage to the proximal His-Purkinje tissue to avoid heart block. This could be very shielding or shunting of the electric field via coating such as MuMETAL® or a non-conductive element so as to add additional safeguards to the conduction system.

In addition to irreversible electroporation—where the tissue is indefinitely destroyed, the catheter systems described herein can also deliver reversible electroporation doses so as to serve as temporary/test doses to ensure the correct tissue is ablated and to add a safety level.

The catheter systems described herein can also be introduced to the LV through puncture of the right aspect of the ventricular septum. The catheter can also be introduced into the LV through transapical access, e.g., by the use of a surgical pericardial window, as well as via epicardial access.

In some embodiments, an expandable balloon is located along one or more of the flexible electrode-carrying elements to enhance stability with tissue.

The catheter systems described herein have the ability to be used a mapping catheter connecting to a 3D mapping system so as to recreate 3D structure of the heart chambers and annotate ventricular signals, His-Purkinje tissues, and 2D/3D dimensions of the heart in combination with fluoroscopy, a navigation system, and intracardiac echocardiography.

The catheter systems described herein can delivery electroporation energy from any electrode, and any pair of electrodes on the same flexible electrode-carrying element of the catheter, adjoining arms, or any combination of a pair of electrodes across the device. This provides for narrow as well as widespread ablation energy delivery.

In some embodiments, the catheter systems can also deliver radiofrequency (RF) ablation energy. This can be in combination with electroporation energy or independent depending on desired tissue, desired depth of energy penetration, and location in the heart.

The electrodes can also have adjacent thermistors or thermocouples to monitor tissue temperature before, during, and after ablation energy delivery.

The electrodes can also have irrigation ports to allow for adequate cooling of the flexible electrode-carrying elements in order to prevent char or coagulum and allow for power delivery and titration as desired. This can be delivered anywhere along the flexible electrode-carrying elements. In addition, this provides not only a port for saline irrigation for cooling, but also for drug delivery.

Ablation can be titrated and combined in to permit safe and effective ablation given a combination of flexible electrode-carrying elements constriction or expansion to allow for small and focused mapping and ablation, as well as large areas of tissue ablation. This includes critical structures in the ventricle, including the ventricular myocardium, proximal and distal His-Purkinje system, and intracavitary dimensions and structures that are complex within the ventricle, including around the mitral valve apparatus, false tendons, papillary muscles, and moderator band.

In some embodiments, the flexible electrode-carrying elements have force sensors at the tips and/or along the lengths of the flexible electrode-carrying elements. This can help determine the amount of contact force the flexible electrode-carrying elements have with the tissue it is in contact with to help guide ablation lesions and durability. In addition to contact force, this can be used in conjugation with a combination of expansion and retraction to add a tissue stretching modality. This can be beneficial because stretching tissue can potentially lower the threshold for electroporation, and thus can be used in combination with/during ablation.

In some embodiments, two such catheter systems, can concurrently be used in separate locations but can work and be placed in tandem.

Another embodiment involving two catheter systems would include that one would be stationary and screwed in, the second would be free floating and able to map, pace, and ablate from either the endocardium, myocardium, epicardial, and vice versa with the first catheter. Another embodiment with the screw-in mechanism would allow for transeptal ablation of myocardium and His-Purkinje tissue.

In some embodiments, the electrodes can be used for pacing at variable outputs across a plurality of electrodes on the same arm, different arms, and/or across varying distances. Using pacing, determination of capture or not permits the ability to determine if the tissue is ablated or still viable. Pacing can be performed both in a unipolar or bipolar fashion.

In some embodiments, the electrodes of the catheter systems described herein are used for impedance-based monitoring during ablation from electrodes to assess for adequate tissue destruction. This can be automated to mark an adequate lesion after, for example, an 8-10 Ohm drop in impedance and marked on the mapping/recording system software (e.g., using a machine learning algorithm to detect and/or predict that a lesion will form as a result of ablation of any type or energy source). Further ablation can be performed if tissue is still able to be captured with pacing.

Aspects of the designs of the catheter systems described herein can also be extended for use in interventional and structural catheterization, temporary pacing and hemodynamic evaluation (contractility), and electrophysiologic ablation procedures such as PVC/VT ablation. For example, given the safety features and utility of the J-tip guidewire and delivery sheath aspects of the catheter systems described herein, such features will be of utility in many complex interventional cardiology procedures, especially those requiring safe navigation for interventions of the heart where valves are required for intervention or repair, transseptal or trans-aortic access is required, these include but are not limited to trans-aortic valve replacement (TAVR), trans-mitral valve replacement (TMVR), trans-tricuspid valve replacement (TTVR), and trans-pulmonic valve replacement (TPVR).

The ability to safely cross the mitral valve and be able to pace may also serve as an all-in-one device in addition to the valve, as these procedures require safe access, but also high-output pacing so that the heart cardiac output is decreased to allow for successful deployment.

The platform of the catheter systems described herein can also be used for safe delivery of an intra-aortic balloon pump. The platform of the catheter systems described herein can also be used for transseptal cannulation for LV venting for cardiopulmonary bypass. The platform of the catheter systems described herein can be used for bedside placement of temporary pacing devices. The platform of the catheter systems described herein can be used for swan-ganz catheter placement at the bedside. The delivery sheath with J-tipped guidewire guidance can also be useful for left atrial appendage occlusion delivery, placement, and re-sealing of leaks. The platform of the catheter systems described herein can be used for assessing and deploying peri-leak devices given this is wire guided and oversheath for easy delivery. The catheter systems described herein can be used for access for renal artery/vein access for renal denervation or access to the splanchnic nerves for HFpEF treatment.

The catheter systems described herein can be used to treat any ventricular dysrhythmias, not only Ventricular Fibrillation. These include but are not limited to all types of premature ventricular contractions (PVCs) and Ventricular Tachycardia.

It should also be understood that the features and usage techniques described herein in relation to the various ablation devices can be combined with the features of other ablation device embodiments and usage techniques described herein. Accordingly, based on such combinations and sub-combinations, an extensive number of ablation device embodiments and usage techniques are envisioned and provided herein.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

1. A multi-functional catheter system comprising:
a delivery sheath defining a first lumen and a longitudinal axis;
a catheter shaft;
a plurality of electrode-carrying elements attached to and distally extending from a distal end portion of the catheter shaft, wherein the catheter shaft and the plurality of electrode-carrying elements are slidably disposable within the first lumen of the delivery sheath and thereby reconfigurable between: (i) a low-profile delivery configuration when the catheter shaft and the plurality of electrode-carrying elements are fully within the first lumen and (ii) a deployed configuration when the plurality of electrode-carrying elements distally extend from a distal end of the first lumen; and
a plurality of electrodes disposed on the plurality of electrode-carrying elements.

2. The multi-functional catheter system of claim 1, wherein the plurality of electrode-carrying elements are configured to self-expand to define a conical shape when in the deployed configuration.

3. The multi-functional catheter system of claim 1, wherein the catheter shaft defines a second lumen.

4. The multi-functional catheter system of claim 3, further comprising a guidewire slidably disposable in the second lumen and distally extendable through and beyond the plurality of electrode-carrying elements.

5. The multi-functional catheter system of claim 1, further comprising at least one electrode attached to the delivery sheath.

6. The multi-functional catheter system of claim 1, further comprising at least one electrode attached to the catheter shaft.

7. The multi-functional catheter system of claim 1, further comprising a plurality of conjoining elements that each extend between distal tips of two electrode-carrying elements of the plurality of electrode-carrying elements to form an electrode loop.

8. The multi-functional catheter system of claim 7, wherein the plurality of conjoining elements are slidably disposed within lumens of the plurality of electrode-carrying elements such that the width or area of the electrode loops are adjustable by tensioning or relaxing the plurality of conjoining elements.

9. A method for treating a patient, the method comprising:
advancing the multi-functional catheter system of any one of claims 1 through 8 into the patient to position the plurality of electrode-carrying elements in a target region; and
energizing at least some of the plurality of electrodes, wherein the energizing provides an energy sufficient for ablation or electroporation of at least some tissue of the target region.

10. The method of claim 9, further comprising stretching, by the plurality of electrode-carrying elements, the at least some tissue of the target region, wherein the stretching occurs simultaneously with the energizing.

11. The method of claim 9, wherein the energizing comprises:
using at least one electrode attached to a single electrode-carrying element of the plurality of electrode-carrying elements as a cathode; and
using at least one other electrode attached to the single electrode-carrying element of the plurality of electrode-carrying elements as an anode.

12. The method of claim 9, wherein the energizing comprises:
using at least one electrode attached to a first electrode-carrying element of the plurality of electrode-carrying elements as a cathode; and
using at least one electrode attached to a second electrode-carrying element of the plurality of electrode-carrying elements as an anode.

13. The method of claim 9, wherein the energizing comprises:
delivering at least one of: RF energy, ultrasound energy, laser energy, light energy, and cryoablation; and
delivering pulsed DC energy.

14. The method of claim 9, further comprising delivering, via one or more of the plurality of electrode-carrying elements suction, irrigation, or a pharmacological agent to the target region.

15. The method of claim 9, wherein the target region is a left or right ventricle of the patient.

16. A method for treating a patient, the method comprising:
delivering electroporation to a target tissue region of the patient by using an electroporation delivery algorithm to deliver one or more ablation sequences to the target tissue region.

17. The method of claim 16, wherein the one or more ablation sequences includes one or more low dose reversible electroporation pulses delivered to the target tissue region to affect superficial Purkinje fibers, and wherein the method includes measuring impedance of the superficial Purkinje fibers after the delivery of the one or more low dose reversible electroporation pulses.

18. The method of claim 17, further comprising delivering an irreversible DC energy to the target tissue region after measuring the impedance.

19. The method of claim 16, wherein a machine learning algorithm is used to monitor the impedance, temperature, and/or electrograms from using ablation electrodes or surrounding electrodes to determine successful ablation of the target tissue region.
